(19) United States
(12) Patent Application Publication
(10) Pub. No.: US 2020/0281138 A1
(43) Pub. Date: Sep. 10, 2020

(54) METHOD OF AUTOMATIC AGRICULTURAL POLLINATION BASED ON MICRO AIR VEHICLE

(71) Applicants: Yi CHEN, Guangdong (CN); Yun LI, Guangdong (CN); Hongnian YU, Guangdong (CN); Dongguan University of Technology, Guangdong (CN)

(72) Inventors: Yi CHEN, Guangdong (CN); Yun LI, Guangdong (CN); Hongnian YU, Guangdong (CN)

(73) Assignees: Dongguan University of Technology, Guangdong (CN); Hongnian YU, Guangdong (CN); Yi CHEN, Guangdong (CN); Yun LI, Guangdong (CN); Hongnian YU, Guangdong (CN)

(21) Appl. No.: 15/774,581

(22) PCT Filed: Apr. 25, 2017
(86) PCT No.: PCT/CN2017/081877

§ 371 Date: May 8, 2018

(30) Foreign Application Priority Data
Jan. 23, 2017 (CN) 201710049073.3

(51) Int. Cl. A01H 1/02; B64C 39/02; B64D 47/08; G05D 1/10; G05D 1/00

(52) U.S. Cl. CPC A01H 1/02; B64C 39/028; B64D 47/08; B64C 2201/141; G05D 1/0094; B64C 2201/127; B64C 2201/12; G05D 1/101

(57) ABSTRACT

The present invention discloses a method of automatic agricultural pollination based on a micro air vehicle, with detailed steps as follows: in a current pollination working area j, a central control system CCS assigning a work task to a micro air vehicle pollinator μMAVi; capturing a data of flower via a camera and a data acquisition device; processing an original data collected from a specific data source, and then screening, filtering and preprocessing the original data; according to a data flow, performing an operation of recognizing flower; assessing whether a designated flower is successfully recognized or not; the micro air vehicle pollinator μMAVi pollinating the designated flower; assessing a pollination result and an effectiveness of the micro air vehicle pollinator μMAVi, and determining whether to end a work of the current working area j and to enter into the next working area j+1 or not. The present invention is more efficient in an automatic process, reducing an operating cost at the same time, being advantageous for propelling intellectualization, and the present invention can provide a higher efficiency and a better pollination quality and improve an agricultural productivity.

### BACKGROUND

### Technical Field

The present invention relates to a method of pollination for plants, and specifically relates to a method of automatic agricultural pollination based on a micro air vehicle (MAV).

### Description of Related Art

“Pollination” is always strongly influencing a correlation both inside and outside an ecological system, a stability of the ecological system and a genetic diversity of phytocoenosium. Research shows that about one third of the food for human beings grows depending on pollination by animals and insects, among which honeybees are one of the main pollinators. In particular, a main reason, the global epidemic of Colony Collapse Disorder (CCD), has seriously influenced the food safety of human beings. The present invention proposes an automatic solution of agricultural pollination by an autonomous micro hummingbird air vehicle.

### SUMMARY OF THE INVENTION

### Technical Problem

An object of the present invention is to provide a method of automatic agricultural pollination based on a micro air vehicle with high efficiency and good effect, in order to solve the problems stated in the above-described background.

### Technical Solution

In order to achieve the above-described object, the present invention provides a technical solution below:

A method of automatic agricultural pollination based on a micro air vehicle, has detailed steps as follows:

(1) in a current pollination working area j, a central control system CCS assigning a work task to a micro air vehicle pollinator μMAVi;

(2) capturing a data of flower via a camera and a data acquisition device;

(3) processing an original data collected from a specific data source, and then screening, filtering and preprocessing the original data;

(4) according to a data flow, performing an operation of recognizing the flower;

(5) assessing whether a designated flower is successfully recognized or not;

(6) the micro air vehicle pollinator μMAVi pollinating the designated flower;

(7) assessing a pollination result and an effectiveness of the micro air vehicle pollinator μMAVi, determining whether to end a work of the current working area j and to enter into the next working area j+1 or not; and

(8) if the assessment being negative, then performing the pollination again; and if the assessment being positive, then entering into the next working area j+1, and repeating steps 1 to 7.

As a further solution of the present invention: the work task in the step (1) comprises pollinating flowers of a specific type of crop, a type of the flowers and a number of the flowers.

As a further solution of the present invention: the data of flower in the step (2) is a photo of the flower.

### Beneficial Effect of the Invention

Comparing with the prior art, the present invention has beneficial effects:

The present invention is more efficient in an automatic process, reducing an operating cost at the same time, beneficial to propelling agricultural intellectualization, and the present invention can provide a higher efficiency and a better pollination quality and improve agricultural productivity.

### BRIEF DESCRIPTION OF THE DRAWINGS

FIG. 1 is a flow diagram of a method process of the present invention.

FIG. 2 is a schematic diagram of a working principle of the present invention.

### DETAILED DESCRIPTION OF THE EMBODIMENT

The technical solution of the present application is described in detail below by combining with specific implementation.

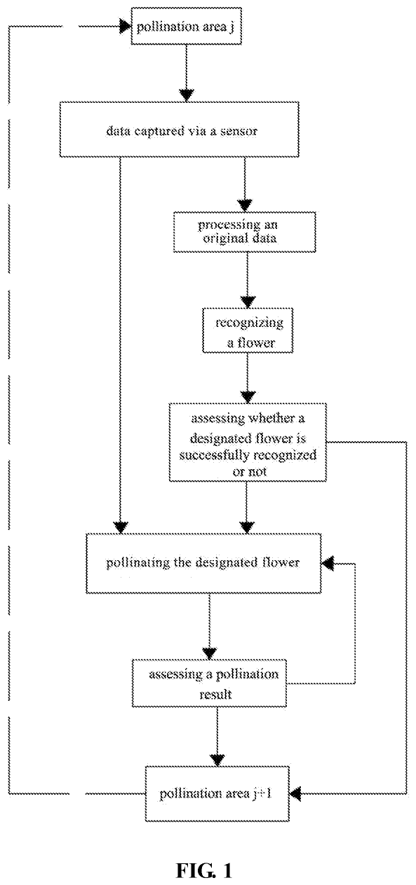

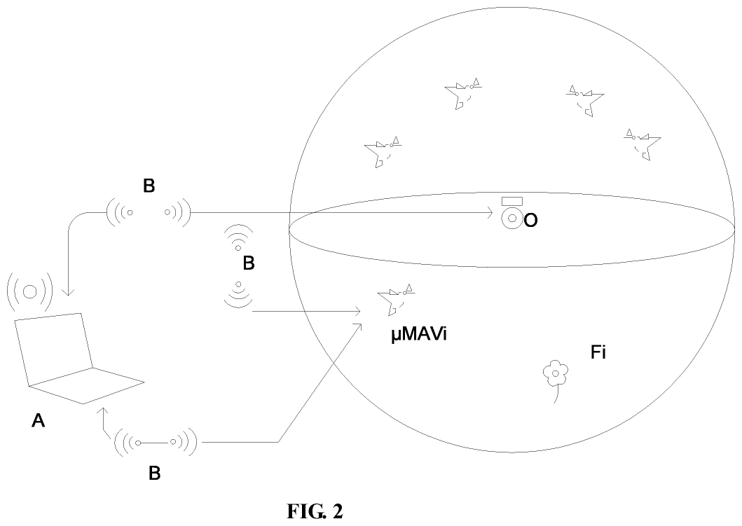

Referring to FIG. 1 and FIG. 2, a method of automatic agricultural pollination based on a micro air vehicle, detailed steps are as follows:

(1) in a current pollination working area j, a task is assigned by a central control system (CCS) to a micro air vehicle pollinator μMAVi; and the task comprises pollinating flowers of a specific type of crop, a type of the flowers and a number of the flowers;

(2) a data of flower is captured via a camera and a data acquisition device; and the data of flower is a photo of the flower;

(3) an original data collected from a specific data source is processed, and then the original data was screened, filtered and preprocessed;

(4) according to a data flow, an operation of recognizing the flower is performed;

(5) whether a designated flower is successfully recognized or not is assessed;

(6) the designated flower is pollinated by the micro air vehicle pollinator μMAVi;

(7) a pollination result and an effectiveness of the micro air vehicle pollinator μMAVi are assessed, whether to end a work of the current working area j and to enter into the next working area j+1 or not is determined; and

(8) if the assessment is negative, then the pollination will be performed again; and if the assessment is positive, then the micro air vehicle pollinator μMAVi will enter into the next working area j+1, and steps 1 to 7 will be repeated.

In application scenarios that require a large-scale pollination activity, such as a production base of grain crop, a botanical garden and an orchard, or a production base of commercial seeds and the like, the method of automatic agricultural pollination based on the micro air vehicle of the present invention provides a “sustainable” food supply method.

A sphere working range is formed with a point O as the center of the camera and an effective working distance of the camera as a radius, μMAVi is a micro air vehicle system and Fi is the flower of the crop.

The invention is more efficient in an automatic process, and reduces an operating cost at the same time, no matter the number of workers increases or decreases. Then agriculture workers, no matter being substituted by other workers or robots, can freely focus on work on which they can exert greater influence and can execute a higher-value work. Besides, requirements for the micro air vehicle for autonomous agricultural pollination create extra jobs, for example maintenance of the micro air vehicle system for autonomous agricultural pollination. Additionally, an agriculture system will more rely on the micro air vehicle for autonomous agricultural pollination, which is advantageous for propelling agricultural intellectualization.

From an economic point of view, the micro air vehicle for autonomous agricultural pollination provides a higher efficiency and a better pollination quality, and improves an agricultural productivity at the same time. The micro air vehicle system for autonomous agricultural pollination will directly influence the grain production, in order to improve a farming efficiency in the future. For example, in 2009, CCD had influenced 24% of grain crop based on a total yield of crop of UK; and in 2013, CCD had influenced products with value of 4 billion dollars in United States. Products of the micro air vehicle for autonomous agricultural pollination will improve development of other industrial products, such as an advanced motor and MEMS system, and manufacturers or suppliers of advanced materials and industrial engineering.

In order to adjust to the requirements for efficient agricultural automatization in the future, the micro air vehicle for autonomous agricultural pollination, as a substitute for natural pollinators, such as honeybee, insect and the like, can act as an artificial pollinator in an industry of intelligent agriculture, reducing a problem that an ecological correlation, protection and stability of an ecological system, a heritable variation of phytocoenosium of crop, and a diversity, a specialization and an evolution of flowers are seriously influenced due to a decrease in a population quantity of natural pollination medium.

The present invention is more efficient in the automatic process, reducing the operating cost at the same time, being advantageous for propelling agricultural intellectualization, and the present invention can provide a higher efficiency and a better pollination quality and improve the agricultural productivity.

The preferred implementation of the present application is described in detail above, but the present application isn't limited by the above-described implementation. Within the scope of knowledge owned by a person skilled in the art, a plurality of variations can also be made without deviating from the spirit of the present application.

1. A method of automatic agricultural pollination based on a micro air vehicle, characterized in that, it has detailed steps as follows:
(1) in a current pollination working area j, a central control system assigning a work task to a micro air vehicle pollinator;
(2) capturing a data of flower via a camera and a data acquisition device;
(3) processing an original data collected from a specific data source, and then screening, filtering and preprocessing the original data;
(4) according to a data flow, performing an operation of recognizing the flower;
(5) assessing whether a designated flower is successfully recognized or not;
(6) the micro air vehicle pollinator pollinating the designated flower;
(7) assessing a pollination result and an effectiveness of the micro air vehicle pollinator, determining whether to end the work of the current working area j and to enter into the next working area j+1 or not; and
(8) if the assessment is negative, then perform the pollination again; and if the assessment is positive, then enter into the next working area j+1, and repeat steps 1 to 7.

2. The method of automatic agricultural pollination based on a micro air vehicle according to claim 1, characterized in that the work task in the step (1) comprises pollinating flowers of a specific type of crop, a type of the flowers and a number of the flowers.

3. The method of automatic agricultural pollination based on a micro air vehicle according to claim 1, characterized in that the data of flower in the step (2) is a photo of the flower.
